(19) United States
(12) Patent Application Publication
(10) Pub. No.: US 2005/0284472 A1
(43) Pub. Date: Dec. 29, 2005

(54) Auto-pumping unit for bag-valve-mask resuscitator

(76) Inventor: Jimmy Lin, Taichung City (TW)

Correspondence Address: BACON & THOMAS, PLLC, 625 SLATERS LANE, FOURTH FLOOR, ALEXANDRIA, VA, 22314, US

(21) Appl. No.: 10/876,459

(22) Filed: Jun. 28, 2004

(51) Int. Cl. A61M 16/00; A62B 9/06

(52) U.S. Cl. 128/202.29

(57) ABSTRACT

An auto-pumping unit for a bag-valve-mask resuscitator includes a housing, a pressure device, and driving mechanism. The housing has a receiving space for accommodating a bag of the bag-valve-mask, and a baffle mounted at a side of the receiving space. The pressure device is movably mounted at the other side of the receiving space opposite to the baffle. The driving mechanism is mounted in the housing and controlled to drive the pressure device to move close to or away from the baffle to compress or release the bag at a predetermined speed.

### BACKGROUND OF THE INVENTION

1. Field of the Invention

The present invention relates to generally to medical first-aid apparatuses, and more specifically to an auto-pumping unit for a bag-valve-mask resuscitator.

2. Description of the Related Art

A conventional bag-valve-mask resuscitator (BVM) is a medical first-aid apparatus commonly used in an emergency case to help the patient breathe. When in use, the mask is covered on the patient's face over the mouse and the nose, and then the rubber ball is compressed at a predetermined frequency to supply the air to the patient to further regain or maintain the patient's life.

During a process of first aid, the most important job to do is to keep the patient breathing. However, when the firs-aid personnel are rarely available on the spot, e.g. generally speaking, only one first-aid person is arranged in the ambulance, it is difficult to control various first-aid apparatuses and take care of the patient at the same time for the first-aid person. When operating a bag-valve-mask resuscitator manually at a stable frequency to supply the air to the patient, the first-aid person may fail to operate other first-aid apparatuses or take care of the patient. This problem is so serious that an improvement is required.

### SUMMARY OF THE INVENTION

The present invention has been accomplished under the circumstances in view. It is the primary objective of the present invention to provide an auto-pumping unit, which automatically operates a bag-valve-mask resuscitator at a stable and accurate frequency to supply the air to the patient. It is a secondary objective of the present invention to provide an auto-pumping unit, which fits different bags having different sizes in a bag-valve-mask resuscitator.

To achieve the foregoing objectives of the present invention, the auto-pumping unit is comprised of a housing having a receiving space for accommodating the bag of the bag-valve-mask, the housing having a baffle mounted at a side of the receiving space, a pressure device movably mounted at the other side of the receiving space, and a driving mechanism mounted in the housing and controlled to drive reciprocating movement of the pressure device at a predetermined speed to further compress the bag at a predetermined frequency.

### BRIEF DESCRIPTION OF THE DRAWINGS

FIG. 1 is a perspective view of a first preferred embodiment of the present invention.

FIG. 2 is a sectional view of the first preferred embodiment of the present invention.

FIG. 3 is a top plain view of the first preferred embodiment of the present invention, showing a big bag in use.

FIG. 4 is similar to FIG. 3, showing the bag compressed.

FIG. 5 is a top plain view of the first preferred embodiment of the present invention, showing that a receiving space is set to fit a small bag.

FIG. 6 is a side sectional view showing a small bag in use.

FIG. 7 is a perspective view of a second preferred embodiment of the present invention.

FIG. 8 is a side sectional view of the second preferred embodiment of the present invention.

FIG. 9 is a perspective view of a third preferred embodiment of the present invention.

FIG. 10 is a side sectional view of the third preferred embodiment of the present invention.

### DETAILED DESCRIPTION OF THE INVENTION

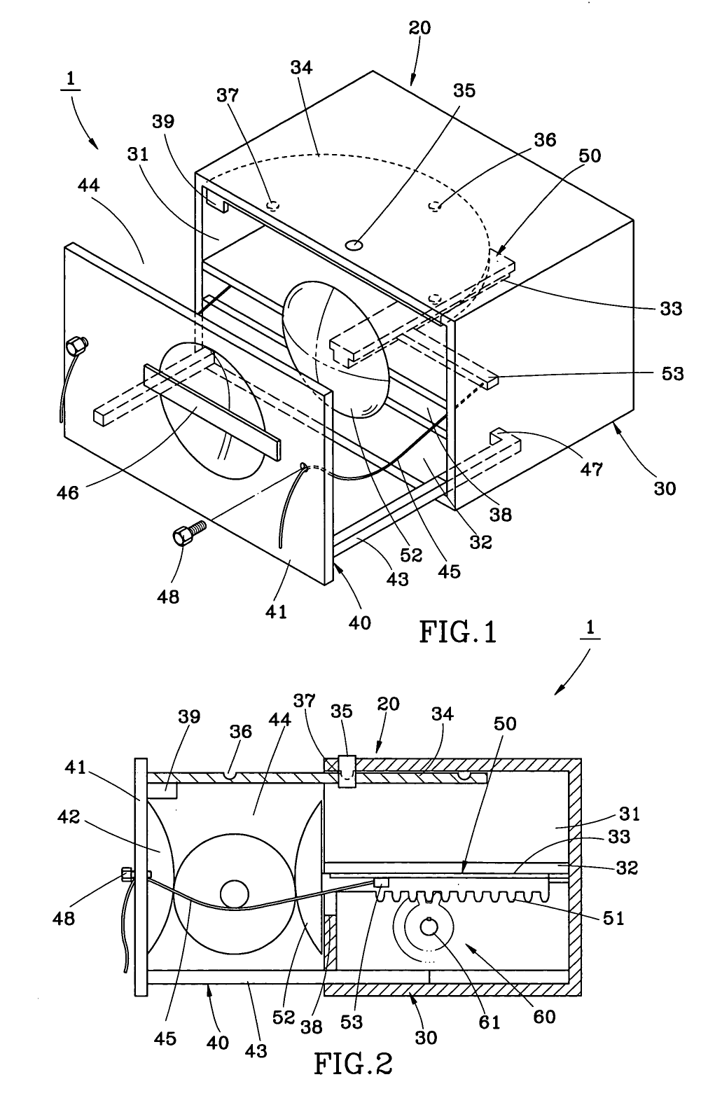

Referring to FIGS. 1 and 2, an auto-pumping unit for a bag-valve-mask resuscitator is constructed in accordance with a first preferred embodiment of the present invention, comprised of a housing 20, a pressure device 50, two cord members 45, and a driving mechanism 60.

The housing 20 includes a main body 30 and a movable part 40. The main body 30 is a hollow rectangular shell having a chamber 31 inside and a seat 32 mounted inside the chamber 31. The seat 32 has an opening 38 and a sliding groove 33. The movable part 40 includes a baffle 41 and two pull bar 43. The baffle 41 has a semispherical stop portion 42 protruding from a midsection of a rear side thereof towards the receiving chamber 31 of the main body 30, a concavity recessed from the midsection of a front side thereof towards the receiving chamber 31, and a handle 46 mounted at the front side thereof and transversely located over the semispherical stop portion 42. The pull bars 43 each have an end connected to a bottom edge of the rear side of the baffle 41 and the other end inserted into the main body 30 and connected to a stop block 47, which prevents the pull bar 43 from slipping off the main body 30. The pull bars 43 are retractable to enable the baffle 41 to be moved close to or away from the main body 30. When the baffle 41 is moved away from the main body 30, a receiving space 44 is defined between the baffle 41 and the main body 30. A semicircular swivel plate 34 is pivoted to a top wall of the main body 30 inside the chamber 31 at a pivot 35. The pivot 35 divides the swivel plate 34 into two halves having different radius of curvature. The swivel plate 34 has a finger block 39 formed at a bottom side thereof for pulling by the user's hand, and a plurality of locating grooves 36 equiangularly arranged on a top side thereof and respectively spaced from the pivot 35 at an equal distance. The main body 30 has two projections 37 protruded downwards from the top wall thereof abutting the top side of the swivel plate 34. The swivel plate 34 can be turned about the pivot 35 in and out of the chamber 31 of the main body 30. When turning the swivel plate 34 out of the chamber 31 of the main body 30 at a predetermined angle, one locating groove 36 of the swivel plate 34 will be forced into engagement with one projection 37 to lock the swivel plate 34 in position. Because the swivel plate 34 can be turned out of the main body 30 selectively at different angles, the swivel plate 34 can be selectively set in one of a series of protruding positions. After locking the swivel plate 34 in one protruding position, the baffle 41 is moved towards the main body 30 and stopped against the protruded swivel plate 34. By means of the aforesaid arrangement, the receiving space 44 between the baffle 41 and the main body 30 is adjustable.

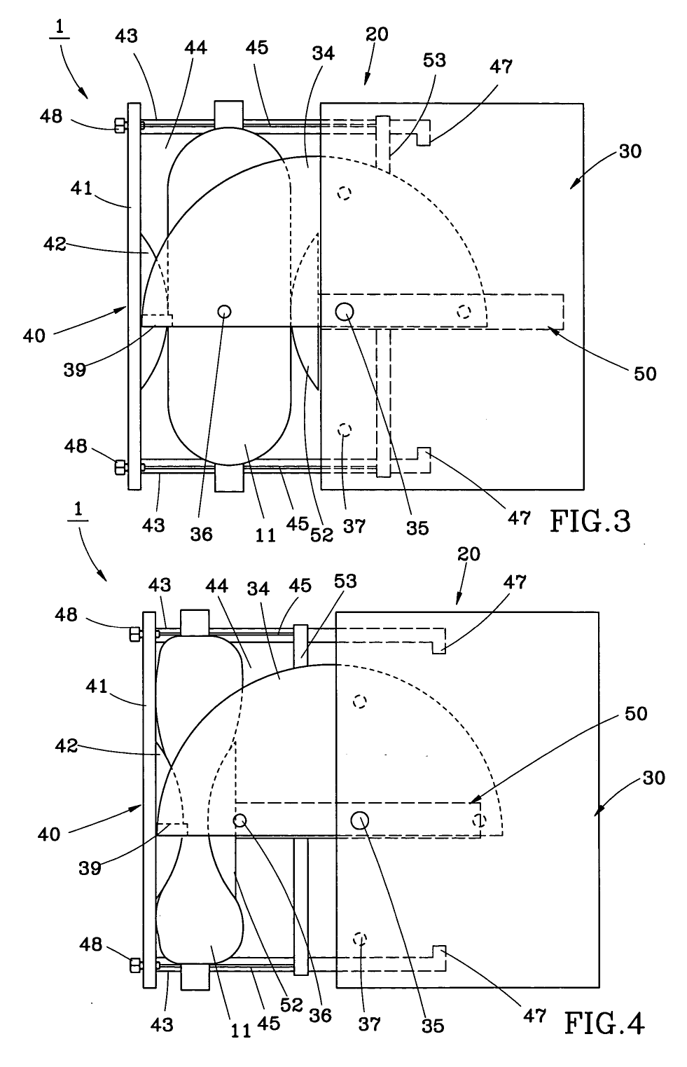

The pressure device 50 includes a rack 51 located at an end thereof and coaxially slidably mounted to the sliding groove 33 of the seat 32, a semispherical press member 52 located at the other end thereof and facing the semispherical stop portion 42, and a cross bar 53 vertically mounted at a bottom side of the rack 51. The pressure device 50 is movable along the sliding groove 33 to enable the press member 52 to move away from the semispherical stop portion 42 for a distance, as shown in FIG. 3, or to move close to the semispherical stop portion 42, as shown in FIG. 4.

The two cord members 45 each have an end respectively fastened to two distal ends of the cross bar 53 and the other end inserted through the baffle 41 of the movable part 40 and fixed thereto by a screw bolt 48. When the receiving space 44 is formed between the main body 30 and the baffle 41, the two cord members 45 are suspended at two sides of the receiving space 44. The user can control the two screw bolts 48 to fix the cord members 45 to the baffle 41 in such a position that the middle point of each cord member 45 is lower than two ends of each cord member 45 respectively connected to the main body 30 and the movable part 40.

The driving mechanism 60 is mounted inside the seat 32 of the housing 20. According to this embodiment, the driving mechanism 60 includes a transmission gear 61 meshed with the rack 51 of the pressure device 50, and a motor (not shown) for driving the rotation of the transmission gear 61 alternately forwards and backwards at a predetermined speed to further reciprocate the rack 51 along the sliding groove 33.

Normally, the bag-valve-mask resuscitator can be made in two sizes for the adult and the children respectively. The size of the bag is also subject to the size of the bag-valve-mask resuscitator. When a big ball 11 is used in the adult-size resuscitator as shown in FIGS. 3 and 4, the swivel plate 34 is turned out of the chamber 31 of the main body 30 at a predetermined angle and pushed against the baffle 41 of the movable part 40 to define the desired receiving space 44 between the movable part 40 and the main body 30 (FIG. 3 shows the relatively bigger half of the swivel plate 34 extended to the outside of the chamber 31 of the main body 30), then the bag 11 of the resuscitator 1 is put in the receiving space 44 and supported with its two ends thereof on the cord members 45. At the same time, the semispherical stop portion 42 of the baffle 41 and the semispherical press member 52 of the pressure device 50 slightly pushes against two sides of the bag 11. Thereafter, the motor of the driving mechanism 60 is started to rotate the transmission gear 61 alternately forwards and backwards to reciprocate the rack 51 along the sliding groove 33 of the seat 32, thereby causing the semispherical press member 52 of the pressure device 50 to compress the bag 11 at a predetermined frequency. Because the semispherical press member 52 of the pressure device 50 is driven to compress the middle part of the bag 11 against the semispherical stop portion 42 of the baffle 41, and the swivel plate 34 is stopped against the top side of the baffle 41, the bag 11 keeps from slipping off the receiving space 44 while being pushed.

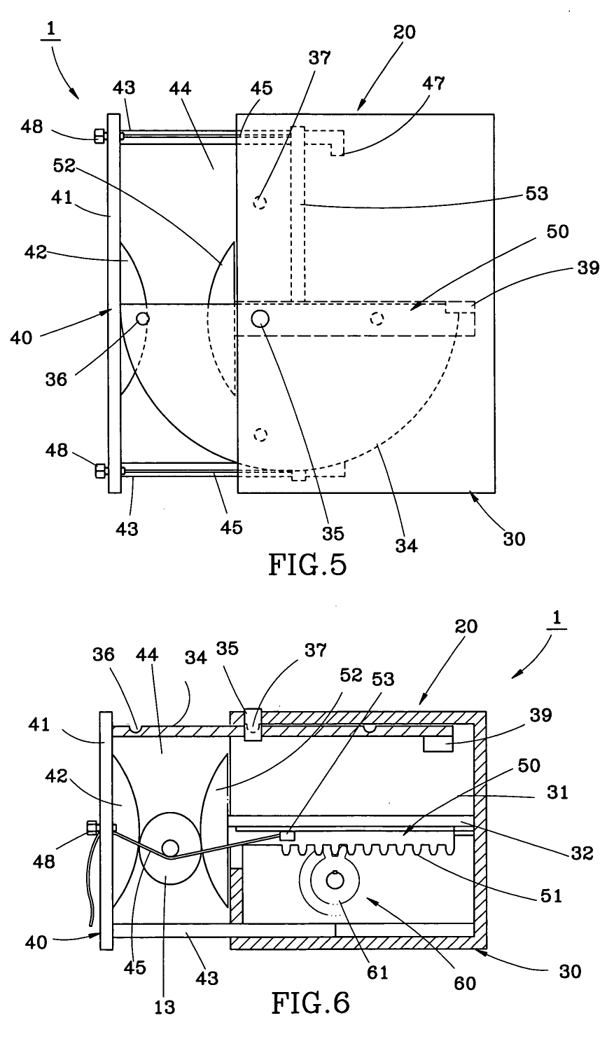

When a small ball 13 is used in the resuscitator for children as shown in FIGS. 5 and 6, the swivel plate 34 is turned out of the chamber 31 of the main body 30 at a predetermined angle and pushed against the baffle 41 of the movable part 40 to define the desired receiving space 44 between the movable part 40 and the main body 30 (FIG. 5 shows the relatively smaller half of the swivel plate 34 extended to the outside of the chamber 31 of the main body 30), then the bag 13 of the resuscitator 1 is put in the receiving space 44 and supported with its two ends thereof on the cord members 45, keeping the semispherical stop portion 42 of the baffle 41 and the semispherical press member 52 of the pressure device 50 slightly pushed against the two sides the bag 11. Thereafter, the motor of the driving mechanism 60 is started to rotate the transmission gear 61 alternately forwards and backwards to reciprocate the rack 51 along the sliding groove 33 of the seat 32, thereby causing the semispherical press member 52 of the pressure device 50 to push against the bag 13 at a predetermined frequency and to supply the air to the patient.

In the aforesaid embodiment, the motor of the driving mechanism 60 obtains the necessary working power from a battery pack (not shown) installed in the seat of the main body 30 of the housing 20. Alternatively, a power cord may be connected to an external power source for supplying power to the motor of the driving mechanism 60. Preferably, a battery power supply and an external power supply are selectively used for the motor of the driving mechanism. Further, the model of the motor is determined subject to the size (type) of the bag-valve-mask resuscitator. Manual switch means may be provided outside the housing 20 for controlling the operation of the motor of the driving mechanism 60. Alternatively, an infrared start switch may be installed in the main body 30 corresponding to the movable member 40 to control the stroke and moving speed of the pressure device 50.

According to the aforesaid embodiment, the invention has the advantages of simple structure and being easy for use. Further, the relative movement between the main body 30 of the housing 20 and the movable part 40 for adjusting the receiving space 44 enables the auto-pumping unit to fit any of a variety of bags for bag-valve-mask resuscitator. The auto-pumping unit automatically pumps the bag stably at predetermined frequency without labor, thereby supplying the air to the patient accurately.

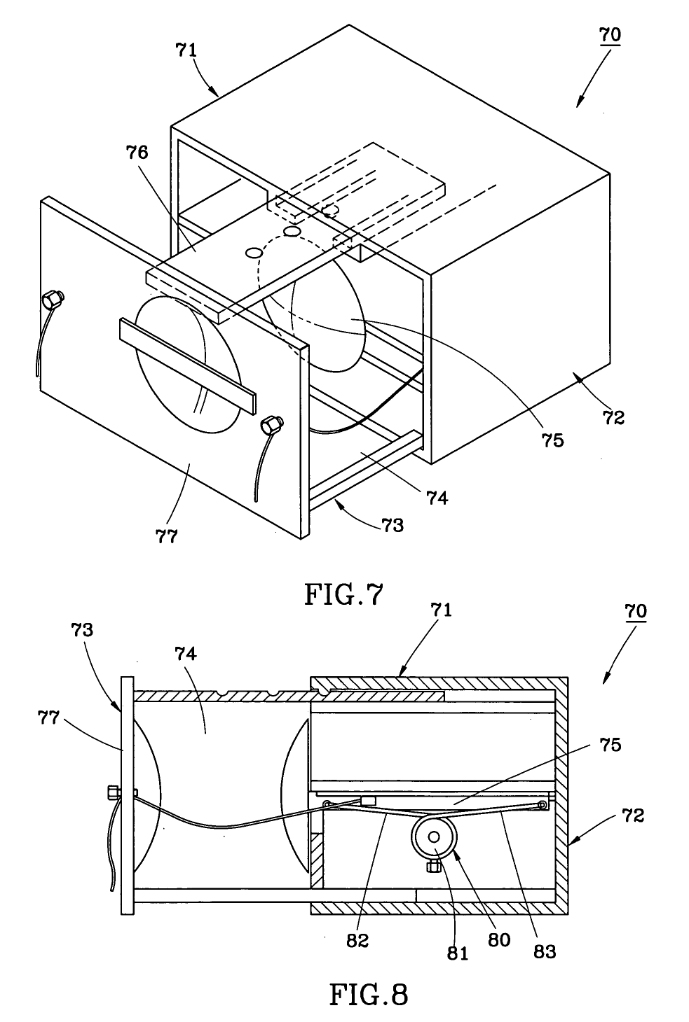

FIGS. 7 and 8 show an auto-pumping unit for use in a bag-valve-mask resuscitator according to a second preferred embodiment of the present invention. According to this design, the auto-pumping unit 70 is substantially similar to the aforesaid first embodiment, comprising a housing 71, a pressure device 75, and a driving mechanism 80. The housing 71 includes a hollow main body 72 and a movable part 73 movable relatively to the hollow main body 72. The hollow main body 72 and the movable part 73 define therebetween a receiving space 74. The pressure device 75 is mounted inside the main body 72 and movable relatively to the movable part 73. The driving mechanism 80 is provided for driving the pressure device 75 relatively to the movable part 73. In this embodiment, an elongated plate member 76 is mounted inside the hollow main body 72 at a top side of the housing 71 and movable in and out of the hollow main body 72. The elongated plate member 76 has an outer end extended out of the hollow main body 72 for stopping against the baffle 77 of the movable part 73 at the desired location. The driving mechanism 80 includes a wheel 81, a first guide rope 82, and a second guide rope 83 (see FIG. 8). The wheel 81 is rotatably mounted inside the main body 72 under the pressure device 75. The first guide rope 82 has an end fixed to a front side of the pressure device 75 and the other end fixed to a right side of the wheel 81. The second guide rope 83 has an end fixed to a rear side of the pressure device 75 and the other end fixed to a left side of the wheel 81. When the wheel 81 is rotated counterclockwise, the wheel 81 rolls up the second guide rope 83 and lets off the first guide rope 82, thereby moving the pressure device 75 toward the movable part 73. On the contrary, when the wheel 81 is rotated clockwise, the wheel 81 rolls up the first guide rope 82 and let off the second guide rope 83, thereby moving the pressure device 75 away from the movable part 73. The driving mechanism 80 accordingly reciprocates the pressure device 75.

The aforesaid second embodiment uses a different design of driving mechanism and a different positioning arrangement between the movable part and the main body to achieve the same effect, fitting any of a variety of bags of different sizes to supply the air to the patient at a stable speed.

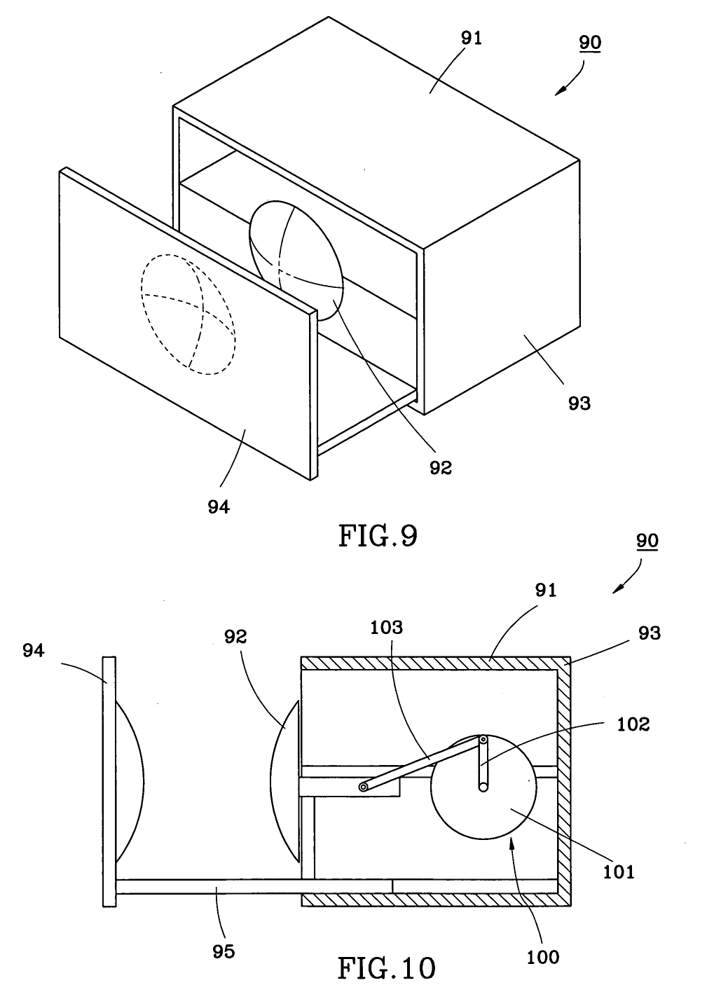

FIG. 9 shows an auto-pumping unit for use in a bag-valve-mask resuscitator according to a third embodiment of the present invention. Substantially similar to the aforesaid first and second preferred embodiments, the auto-pumping unit 90 of this third embodiment includes a housing 91, a pressure device 92, and a driving mechanism 100. The main features of this third embodiment are recited hereinafter. An elongated retractable member 95 is mounted between the main body 93 of the housing 91 and the movable part 94. The elongated retractable member 95 has an end connected to the bottom side of the movable part 94 and the other end inserted into the inside of the main body 93 and positioned in the main body 93. By means of the elongated retractable member 95, the movable part 94 is moved away from or close to the main body 93.

The driving mechanism 100 includes a wheel 101, a first link 102, and a second link 103. The first link 102 has a first end pivoted to an axis of the wheel 101 and a second end connected to a periphery of the wheel 101. The second link 103 has an end pivoted to the second end of the first link 102 and the other end pivoted to the rear end of the press member 92. Therefore, rotating the wheel 101 moves the press member 92 back and forth.

Further, another press member may be used to as a substitute of the semispherical stop portion of the movable part and driven by a driving mechanism to move relatively to the press member of the pressure device to compress/release the bag.

1. An auto-pumping unit for automatically compressing a ball of a bag-valve-mask resuscitator, comprising:
a housing having a receiving space for accommodating said bag and a baffle mounted at a side of said receiving space for stopping against said bag within said receiving space;
a pressure device movably mounted at a side of said receiving space opposite to said baffle for moving close to or away from said baffle; and
a driving mechanism mounted in said housing for driving reciprocating movement of said pressure device at a predetermined speed to further compress said bag at a predetermined frequency.

2. The auto-pumping unit as defined in claim 1 further comprising two cord members, said two cord members being mounted in said housing at two opposite sides of said receiving space for support two distal ends of said bag within said receiving space.

3. The auto-pumping unit as defined in claim 1, wherein said driving mechanism drives said pressure device to reciprocate linearly.

4. The auto-pumping unit as defined in claim 3, wherein said pressure device comprises a rack, and the driving mechanism comprises a transmission gear meshed with said rack and rotated to drive said rack to reciprocate.

5. The auto-pumping unit as defined in claim 3, wherein said driving mechanism comprises a wheel, a first guide rope, and a second guide rope, said wheel being mounted in said housing, said first guide rope being fixedly connected between a front side of said pressure device and a right side of said wheel, said second guide rope being connected between a rear side of said pressure device and a left side of said wheel, said wheel being rotated to drive said pressure device to reciprocate.

6. The auto-pumping unit as defined in claim 3, wherein said driving mechanism comprises a wheel, a first link, and a second link, said wheel being rotatably mounted in said housing, said first link having a first end pivoted to an axis of said wheel and a second end connected to a periphery of said wheel, said second link having a first end pivoted to the second end of said first link and a second end pivoted to the rear end of said pressure device for enabling reciprocating movement of said pressure device by rotation of said wheel.

7. The auto-pumping unit as defined in claim 1, wherein said housing comprises a main body and a movable part movable relatively to said main body and positioned at a predetermined location; said baffle is located on said movable part; said receiving space being located between said movable part and said main body.

8. The auto-pumping unit as defined in claim 7, wherein said main body of said housing comprises a semicircular swivel plate pivotally mounted inside said main body on a pivot of a top side of said main body, said pivot dividing said swivel plate into two halves having different radius of curvature, said swivel plate having a finger block at a bottom side thereof for pulling by a hand to turn said swivel plate out of said main body for stopping against said movable part for enabling said bag to be set in said receiving space between said movable part and said main body.

9. The auto-pumping unit as defined in claim 7, wherein said housing comprises an elongated plate member mounted at an inner top side of said hollow main body and movable in and out of said hollow main body, said elongated plate member having an outer end extended out of said hollow main body for stopping against said movable part for enabling said bag to be set in said receiving space between said movable part and said main body.

10. The auto-pumping unit as defined in claim 7, wherein said housing comprises an elongated retractable member connected between said main body and a bottom side of said movable part and movable to drive said movable part to move close to or away from said main body.

11. An auto-pumping unit for automatically compressing a ball of a bag-valve-mask resuscitator, comprising:
a housing having a receiving space for accommodating said bag;
two pressure devices movably mounted respectively at two sides of said receiving space for moving close to or away from each other therebetween; and
a driving mechanism mounted in said housing for driving said two pressure devices to reciprocate close to or away from each other at a predetermined speed.
